(19) United States
(12) Patent Application Publication
(10) Pub. No.: US 2024/0071616 A1
(43) Pub. Date: Feb. 29, 2024

(54) SYSTEMS AND METHODS TO IMPROVE THERAPEUTIC OUTCOMES

(71) Applicant: Micronoma, Inc., San Diego, CA (US)

(72) Inventors: Eddie ADAMS, San Diego, CA (US); Sandrine MILLER-MONTGOMERY, San Diego, CA (US)

(21) Appl. No.: 18/252,706

(22) PCT Filed: Nov. 11, 2021
(86) PCT No.: PCT/US2021/058999

§ 371 Date: May 11, 2023

Related U.S. Application Data
(60) Provisional application No. 63/112,972, filed on Nov. 12, 2020.

(51) Int. Cl. G16H 50/20; G16B 20/00; G16H 50/30

(52) U.S. Cl. CPC G16H 50/20; G16B 20/00; G16H 50/30

(57) ABSTRACT

Provided are system and methods to improve therapeutic agent outcomes based on microbial and/or non-microbial nucleic acid compositions.

### CROSS-REFERENCE

This application claims benefit of U.S. Provisional Patent Application No. 63/112,972, filed Nov. 12, 2020, which is entirely incorporated herein by reference.

### BACKGROUND

Pharmacogenomics is the practice of utilizing genomic data from a subject to determine suitability or likely efficacy of a therapeutic intervention prior to administration to the subject. Prior to administering a new therapeutic agent, a subject's genome that encodes for enzymes that metabolize a given therapeutic are sequenced and analyzed to determine whether an enzymatic mutation is present that would place the subject at risk of pharmaceutical toxicity or provide a potentially beneficial kinetic profile for metabolizing a given compound. Without pharmacogenomics testing, therapeutic risk or benefit of a given compound is unknown and potentially poorly targeted for a population with diverse genomic data. In addition to surveying a subject's individual mutations in drug metabolizing enzymes, pharmacogenomics seeks to integrate a host of other clinical and non-clinical measurements or reporters with a subject's underlying genetic make-up to arrive at an integrative understanding of the factors that may contribute to drug's in-vivo performance and safety. Such factors include but are not limited to sunlight, infection, disease, occupational exposures, psychologic status, dietary factors, cardiovascular function, gastrointestinal function, immunologic function, stress, starvation and more. However, absent from such factors is the contribution of a subject's various microbiomes and the potential impact a subject's microbiome may have on a subject's biology. Therefore, there is an unmet need for methods to identify the impact a subject's microbiome could have on pharmaceutical therapeutic agents.

### SUMMARY

Provided herein are systems and methods of their use to prospectively or retrospectively determine the therapeutic outcome of a given compound for a given subject. In some embodiments, the administered therapeutic may treat a subject's cancer. In some embodiments, the subjects are a human or other non-human mammal. In some embodiments, the microbiome of a given subject may be analyzed to determine the influence of the microbiome on the metabolism of a given compound towards an enhanced kinetic profile or pharmaceutical toxicity. In some embodiments, the microbiome of a given individual may be unique with respect to the cancer treated. In some embodiments, the microbiome may be screened by isolating cell-free microbial nucleic acid compositions of DNA or RNA from a subject's liquid biopsy. In some embodiments, the microbial nucleic acid composition may be determined by nucleic acid sequencing techniques.

Aspects of the disclosure provided herein, in some embodiments, comprise a method for generating a predictive model, comprising: (a) receiving: (i) one or more liquid biopsies of a first set of one or more subjects, wherein said one or more liquid biopsies comprise one or more microbial and non-microbial nucleic acid compositions; and (ii) at least one therapeutic outcome of said first set of one or more subjects administered a therapeutic treatment; (b) determining one or more sequences of said one or more microbial and non-microbial nucleic acid compositions of said first set of one or more subjects; (c) determining a correlation between said first set of one or more subjects' said at least one therapeutic outcome and said one or more sequences of said one or more microbial and non-microbial nucleic acid compositions; and (d) generating a predictive model with said correlation, wherein said predictive model is configured to provide a prediction of at least one therapeutic outcome of a second set of one or more subjects when administered said therapeutic treatment based on an input to said predictive model of said second set of one or more subjects' one or more microbial and non-microbial nucleic acid composition one or more sequences, wherein said first set of one or more subjects is different than said second set of one or more subjects. In some embodiments, the first or second set of one or more subjects' said at least one therapeutic outcome comprises therapeutic efficacy, therapeutic failure, therapeutic safety, therapeutic adverse side effect or any combination thereof. In some embodiments, the first or second set of one or more subjects' one or more microbial nucleic acid compositions comprise cell-free (cf) nucleic acids. In some embodiments, the first or second set of one or more subjects' one or more microbial nucleic acid compositions comprise microbial cell-free microbial DNA (cf-mbDNA), cell-free microbial RNA (cf-mbRNA) or any combination thereof. In some embodiments, the first or second set of one or more subjects' one or more microbial nucleic acid compositions comprise an origin of viral, bacterial, archaeal, fungal sources or any combination thereof. In some embodiments, the therapeutic treatment treats cancer. In some embodiments, the predictive model is used to analyze a therapeutic outcome of subjects treated for acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, or uveal melanoma or any combination thereof. In some embodiments, the predictive model comprises an artificial intelligence machine learning model, wherein said artificial intelligence machine learning model is trained with said one or more microbial and non-microbial nucleic acid composition said one or more sequences of said first set of one or more subjects and said correlation between said first set of one or more subjects' said one or more microbial and non-microbial nucleic acid composition said one or more sequences and said at least one therapeutic outcome. In some embodiments, the therapeutic outcome of said second set of one or more subjects is used to triage therapeutic clinical trial subjects into responder, non-responder, non-adverse, and adverse groups prior to therapeutic intervention. In some embodiments, the predictive model is used retrospectively. In some embodiments, the second set of one or more subjects' said at least one therapeutic outcome of said predictive model is utilized to longitudinally model the course of one or more cancers' response to therapy. In some embodiments, the first or second set of one or more subjects' one or more microbial or non-microbial nucleic acid compositions comprise one or more single nucleotide polymorphisms (SNPs), insertions and/or deletions (INDELS), genomic amplifications and rearrangements or any combination thereof. In some embodiments, the first or second set of one or more subjects' one or more non-microbial nucleic acid compositions comprise cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA and/or methylation patterns of circulating tumor cell derived RNA, or any combination thereof. In some embodiments, receiving further comprises said first set of one or more subjects' non-genomic data comprising gender, age, weight, body mass index, dietary factors, cardiovascular function, gastrointestinal function, immunological function, liver function, renal function, albumin concentration, alcohol intake, tobacco or marijuana use, pregnancy, lactation, exercise, comorbidities, occupational exposures, psychological status, other medications, or any combination thereof. In some embodiments, the predictive model is a machine learning model. In some embodiments, the predictive model is a regularized machine learning model. In some embodiments, the predictive model is a combination of one or more machine learning models. In some embodiments, the predictive model identifies and removes said first or second one or more subjects' one or more microbial or non-microbial nucleic acid compositions classified as noise while selectively retaining other said one or more microbial or non-microbial features termed signal. In some embodiments, the first or second set of one or more subjects are non-human mammal. In some embodiments, the first or second set of one or more subjects are human. In some embodiments, the said liquid biopsy comprises whole blood, plasma, serum, saliva, sputum, urine, cerebral spinal fluid, sweat, tears, exhaled breath condensate, stool, or any combination thereof.

Another aspect of the disclosure described herein, in some embodiments, comprises a method for generating a therapeutic treatment prediction of one or more subjects, comprising: (a) providing one or more biological samples of a first set of one or more subjects, wherein said first set of one or more subjects comprise a therapeutic treatment outcome when administered a therapeutic treatment; (b) sequencing said one or more microbial and non-microbial nucleic acid compositions of said first set of one or more subjects thereby generating one or more sequences; (c) training a predictive model with said first set of one or more subjects' microbial and non-microbial nucleic acid compositions said one or more sequences and said therapeutic treatment outcome, thereby producing a trained predictive model; and (d) generating a therapeutic treatment prediction for a second set of one or more subjects by inputting said second set of one or more subjects' one or more microbial and non-microbial nucleic compositions one or more sequences, clinical meta data, and said therapeutic treatment to be administered into said predictive model. In some embodiments, the therapeutic treatment treats cancer. In some embodiments, the first or second set of one or more subjects' one or more microbial nucleic acid compositions comprise cell-free (cf) nucleic acids. In some embodiments, the first or second set of one or more subjects' one or more microbial nucleic acid compositions comprise microbial cell-free microbial DNA (cf-mbDNA), cell-free microbial RNA (cf-mbRNA), or any combination thereof. In some embodiments, the first or second set of one or more subjects' one or more microbial nucleic acid compositions originate from viral, bacterial, archaeal, fungal sources or any combination thereof. In some embodiments, the therapeutic treatment prediction is used to triage therapeutic clinical trial subjects into responder, non-responder, non-adverse, and adverse groups prior to therapeutic intervention. In some embodiments, the trained predictive model is used retrospectively. In some embodiments, the trained predictive model is utilized to longitudinally model the course of one or more cancers' response to therapy. In some embodiments, the predictive model is used to analyze said therapeutic treatment prediction of one or more subjects receiving treatment for acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, or uveal melanoma or any combination thereof. In some embodiments, the predictive model comprises a machine learning model. In some embodiments, the machine learning model is a regularized machine learning model. In some embodiments, the machine learning model is a combination of one or more machine learning models. In some embodiments, the predictive model identifies and removes said first or second one or more subjects' one or more microbial or non-microbial nucleic acids classified as noise while selectively retaining other said one or more microbial or non-microbial features termed signal. In some embodiments, the first or second set of one or more subjects are non-human mammal. In some embodiments, the first or second set of one or more subjects are human. In some embodiments, the one or more biological samples are liquid biopsies. In some embodiments, the one or more biological samples is whole blood, plasma, serum, saliva, sputum, urine, cerebral spinal fluid, sweat, tears, exhaled breath condensate, stool or any combination thereof. In some embodiments, the first or second set of one or more subjects' treatment outcome comprise efficacy, failure, safety, adverse side effects, or any combination thereof. In some embodiments, the method further comprising concentrating said first or second set of one or more subjects' one or more microbial and non-microbial nucleic acid compositions of said first or second set of subjects' one or more biological samples.

Another aspect of the disclosure described herein, in some embodiments, comprises a method of generating a predictive model, comprising: (a) receiving: (i) one or more liquid biopsies of one or more subjects comprising one or more microbial nucleic acid compositions; and (ii) at least one therapeutic outcome of said one or more subjects undergoing treatment in a clinical trial; (b) analyzing one or more sequences of said one or more microbial nucleic acid compositions of said one or more subjects; and (c) generating a predictive model with said one or more sequences of said one or more microbial nucleic acid compositions and said at least one therapeutic outcome of said one or more subjects. In some embodiments, the at least one therapeutic outcome comprises therapeutic efficacy, therapeutic failure, therapeutic safety, therapeutic adverse side effect, or any combination thereof. In some embodiments, the one or more microbial nucleic acid compositions comprise cell-free (cf) nucleic acids. In some embodiments, the one or more microbial nucleic acid compositions comprise microbial cell-free microbial DNA (cf-mbDNA), cell-free microbial RNA (cf-mbRNA), or any combination thereof. In some embodiments, the one or more microbial nucleic acid compositions comprise an origin of viral, bacterial, archaeal, fungal sources, or any combination thereof. In some embodiments, the treatment treats cancer. In some embodiments, the predictive model is configured to analyze said therapeutic outcome of subjects treated for acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, or uveal melanoma, or any combination thereof. In some embodiments, the predictive model is configured to triage therapeutic clinical trial subjects into responder, non-responder, non-adverse, and adverse groups prior to therapeutic intervention. In some embodiments, the predictive model is used retrospectively. In some embodiments, the predictive model is configured to provide at least one therapeutic outcome prediction in response to a second set of subjects' one or more microbial nucleic acid compositions' one or more sequences to longitudinally model the course of one or more cancers' response to said treatment. In some embodiments, the one or more liquid biopsies further comprise non-microbial nucleic acid compositions, wherein said one or more microbial or non-microbial nucleic acid compositions comprise one or more single nucleotide polymorphisms (SNPs), insertions and/or deletions (INDELS), genomic amplifications and rearrangements or any combination thereof. In some embodiments, the one or more non-microbial nucleic acid compositions comprise cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA and/or methylation patterns of circulating tumor cell derived RNA or any combination thereof. In some embodiments, receiving further comprises said one or more subjects' non-genomic data comprising gender, age, weight, body mass index, dietary factors, cardiovascular function, gastrointestinal function, immunological function, liver function, renal function, albumin concentration, alcohol intake, tobacco or marijuana use, pregnancy, lactation, exercise, comorbidities, occupational exposures, psychological status, other medications, or any combination thereof. In some embodiments, the predictive model is a machine learning model. In some embodiments, the predictive model is a regularized machine learning model. In some embodiments, the predictive model is a combination of one or more machine learning models. In some embodiments, the predictive model identifies and removes said one or more subjects' one or more microbial nucleic acid compositions classified as noise while selectively retaining other said one or more microbial features termed signal. In some embodiments, the one or more subjects are non-human mammal. In some embodiments, the one or more subjects are human. In some embodiments, the one or more liquid biopsies comprise whole blood, plasma, serum, saliva, sputum, urine, cerebral spinal fluid, sweat, tears, exhaled breath condensate, stool, or any combination thereof. In some embodiments, the method further comprises determining a correlation between said one or more subjects' said at least one therapeutic outcome and said one or more sequences of said one or more microbial or non-microbial nucleic acid compositions.

Another aspect of the disclosure described herein, in some embodiments, comprises a computer-implemented method for utilizing a predictive model to provide a therapeutic treatment prediction for one or more subjects, the method comprising: (a) receiving a first set of one or more subjects' one or more liquid biopsies comprising one or more non-microbial and microbial nucleic acid compositions genetic sequences and corresponding at least one therapeutic outcome of said first set of one or more subjects' when exposed to a treatment; (b) training a predictive model with said genetic sequences and said corresponding therapeutic responses of said first set of one or more subjects, thereby generating a trained predictive model; and (c) outputting a therapeutic treatment prediction using said trained predictive model when inputted with a second set of one or more subjects' microbial and non-microbial nucleic acid sequences and corresponding treatment to be administered. In some embodiments, the first or second set of one or more subjects' at least one therapeutic outcome comprises therapeutic efficacy, therapeutic failure, therapeutic safety, therapeutic adverse side effect or any combination thereof. In some embodiments, the first or second set of one or more subjects' one or more microbial nucleic acid compositions comprise cell-free (cf) nucleic acids. In some embodiments, the first or second set of one or more subjects' one or more microbial nucleic acid compositions comprise microbial cell-free microbial DNA (cf-mbDNA), cell-free microbial RNA (cf-mbRNA) or any combination thereof. In some embodiments, the first or second set of one or more subjects' one or more microbial nucleic acid compositions comprise an origin of viral, bacterial, archaeal, fungal sources or any combination thereof. In some embodiments, the treatment treats cancer. In some embodiments, the predictive model is configured to analyze a therapeutic outcome of subjects treated for acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, or uveal melanoma or any combination thereof. In some embodiments, the predictive model is configured to triage therapeutic clinical trial subjects into responder, non-responder, non-adverse, and adverse groups prior to therapeutic intervention. In some embodiments, the predictive model is used retrospectively. In some embodiments, the second set of one or more subjects' at least one therapeutic outcome of said predictive model is utilized to longitudinally model the course of one or more cancers' response to therapy. In some embodiments, the first or second set of one or more subjects' microbial or non-microbial nucleic acid compositions comprise one or more single nucleotide polymorphisms (SNPs), insertions and/or deletions (INDELS), genomic amplifications and rearrangements, or any combination thereof. In some embodiments, the first or second set of one or more subjects' one or more non-microbial nucleic acid compositions comprise cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA and/or methylation patterns of circulating tumor cell derived RNA, or any combination thereof. In some embodiments, receiving further comprises said first set of one or more subjects' non-genomic data comprising gender, age, weight, body mass index, dietary factors, cardiovascular function, gastrointestinal function, immunological function, liver function, renal function, albumin concentration, alcohol intake, tobacco or marijuana use, pregnancy, lactation, exercise, comorbidities, occupational exposures, psychological status, other medications, or any combination thereof. In some embodiments, the predictive model is a machine learning model. In some embodiments, the predictive model is a regularized machine learning model. In some embodiments, the predictive model is a combination of one or more machine learning models. In some embodiments, the predictive model identifies and removes said first or second set of one or more subjects' one or more microbial nucleic acid compositions classified as noise while selectively retaining other said one or more microbial features termed signal. In some embodiments, the first or second set of one or more subjects are non-human mammal. In some embodiments, the first or second set of one or more subjects are human. In some embodiments, the one or more liquid biopsies comprises whole blood, plasma, serum, saliva, sputum, urine, cerebral spinal fluid, sweat, tears, exhaled breath condensate, stool or any combination thereof. In some embodiments, the method further comprises determining a correlation between said first set of one or more subjects' said at least one therapeutic outcome and said one or more sequences of said one or more microbial and non-microbial nucleic acid compositions.

In some embodiments, a correlation between a subject's microbial nucleic acid composition and therapeutic outcome may be developed and implemented in a predictive model. In some embodiments, the correlation is developed between the therapeutic outcome of a given compound for one or more subjects and their respective one or more microbial nucleic acid compositions. In some embodiments, the predictive model may be a machine learning model or an ensemble of machine learning models. In some embodiments, the predictive model may predict prospective or retrospective therapeutic outcomes of a given compound for a given subject based on their one or more microbial nucleic acid compositions of DNA or RNA.

Aspects disclosed herein provide a method for a clinical study data analysis system, comprising: (a) receiving (i) a liquid biopsy comprising one or more microbial nucleic acid compositions; and (ii) at least one therapeutic outcome from one or more subjects undergoing treatment in a clinical trial; and (b) analyzing the sequence of the one or more microbial nucleic acid compositions of the one or more subjects; (c) determining a correlation between the one or more subjects' at least one therapeutic outcome and the sequence of the one or more microbial nucleic acid compositions; and (d) generating a predictive model with the correlation where the predictive model provides at least one therapeutic outcome of the one or more microbial nucleic acid compositions of the one or more subjects. In some embodiments, receiving further comprises non-genomic data comprising one or more subjects' gender, age, weight, body mass index, dietary factors, cardiovascular function, gastrointestinal function, immunological function, liver function, renal function, albumin concentration, alcohol intake, tobacco or marijuana use, pregnancy, lactation, exercise, comorbidities, occupational exposures, psychological status, other medications or any combination thereof. In some embodiments, at least one therapeutic outcome comprises therapeutic efficacy, therapeutic failure, therapeutic safety, therapeutic adverse side effect or any combination thereof. In some embodiments, the one or more microbial nucleic acid compositions comprise cell-free (cf) nucleic acids. In some embodiments, the one or more microbial nucleic acid compositions comprise microbial cell-free microbial DNA (cfDNA), cell-free microbial RNA (cfRNA) or any combination thereof. In some embodiments, the one or more microbial nucleic acid compositions comprise an origin of viral, bacterial, archaeal, fungal or any combination thereof. In some embodiments, the treatment is for cancer. In some embodiments, the predictive model is used to analyze the therapeutic outcome of subjects treated for acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma or any combination thereof. In some embodiments, the predictive model comprises an artificial intelligence machine learning model where the artificial intelligence machine learning model is trained with the one or more microbial nucleic acid compositions of the one or more subjects and the correlation between the one or more subjects' one or more microbial nucleic acid compositions and at least one therapeutic outcome. In some embodiments, the predictive model of at least one therapeutic outcome of the one or more subject is used to triage therapeutic clinical trial subjects into responder, non-responder, non-adverse, and adverse groups prior to therapeutic intervention. In some embodiments, the predictive model is used retrospectively. In some embodiments, the at least one therapeutic outcome of the predictive model is utilized to longitudinally model the course of one or more cancers' response to therapy. In some embodiments, the one or more microbial nucleic acid compositions comprise non-microbial nucleic acid composition comprising one or more single nucleotide polymorphisms (SNPs), insertions and/or deletions (INDELS), genomic amplifications and rearrangements or any combination thereof. In some embodiments, the one or more microbial nucleic acid compositions comprise cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA and/or methylation patterns of circulating tumor cell derived RNA or any combination thereof. In some embodiments, the predictive model is a machine learning model. In some embodiments, the predictive model is a regularized machine learning model. In some embodiments, the predictive model is a combination of one or more machine learning models. In some embodiments, the predictive model identifies and removes the one or more microbial nucleic acid compositions classified as noise while selectively retaining other one or more microbial features termed signal. In some embodiments, the one or more subjects are non-human mammal. In some embodiments, the one or more subjects are human. In some embodiments, the liquid biopsy comprises whole blood, plasma, serum, saliva, sputum, urine, cerebral spinal fluid, sweat, tears, exhaled breath condensate, stool or any combination thereof.

Aspects disclosed herein provide a method for a therapeutic treatment study data analysis system, comprising: (a) providing one or more biological samples of one or more subjects wherein a therapeutic treatment outcome of efficacy is demonstrated; (b) providing one or more biological samples of one or more subjects wherein a therapeutic treatment outcome of failure is demonstrated; (c) providing one or more biological samples of one or more subjects wherein a therapeutic treatment outcome of safety is demonstrated; (d) providing one or more biological samples of one or more subjects wherein a therapeutic treatment outcome of adverse side effects is demonstrated; (e) concentrating a one or more microbial nucleic acid composition of said one or more subjects' one or more biological sample; (f) analyzing said one or more microbial nucleic acid compositions of said one or more subjects; (g) determining a correlation between the subjects treatment outcome and the nucleic acid compositions; (h) training an artificial intelligence with the microbial nucleic acid compositions of subjects and the correlation between the subject's microbial nucleic acid compositions and the therapeutic treatment outcome; and (i) generating a treatment outcome predictive model outcome with the trained artificial intelligence. In some embodiments, the therapeutic treatment is to treat cancer. In some embodiments, the microbial nucleic acid compositions comprise cell-free (cf) nucleic acids. In some embodiments, the microbial nucleic acid compositions comprise microbial cell-free microbial DNA (cfDNA), cell-free microbial RNA (cfRNA) or any combination thereof. In some embodiments, the one or more microbial nucleic acid compositions originate from viral, bacterial, archaeal, fungal origin or any combination thereof. In some embodiments, the treatment outcome predictive model of said treatment outcome of said unknown subject is used to triage therapeutic clinical trial subjects into responder, non-responder, non-adverse, and adverse groups prior to therapeutic intervention. In some embodiments, the treatment outcome predictive model is used retrospectively. In some embodiments, the treatment outcome predictive model is utilized to longitudinally model the course of one or more cancers' response to therapy. In some embodiments, the treatment outcome predictive model is used to analyze the therapeutic outcome of subjects receiving treatment for acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma or any combination thereof. In some embodiments, the artificial intelligence is a machine learning model. In some embodiments, the artificial intelligence is a regularized machine learning model. In some embodiments, the artificial intelligence is a combination of one or more machine learning models. In some embodiments, the treatment outcome predictive model identifies and removes one or more microbial nucleic acids classified as noise while selectively retaining other one or more microbial features termed signal. In some embodiments, the one or more subjects are non-human mammal. In some embodiments, the one or more subjects are human. In some embodiments, the one or more biological samples is a liquid biopsy. In some embodiments, the one or more biological samples is whole blood, plasma, serum, saliva, sputum, urine, cerebral spinal fluid, sweat, tears, exhaled breath condensate, stool or any combination thereof.

Aspects disclosed herein provide a method for a therapeutic treatment study data analysis system, comprising: (a) providing one or more biological samples of one or more subjects wherein a therapeutic treatment outcome of efficacy is demonstrated; (b) providing one or more biological samples of one or more subjects wherein a therapeutic treatment outcome of failure is demonstrated; (c) providing one or more biological samples of one or more subjects wherein a therapeutic treatment outcome of safety is demonstrated; (d) providing one or more biological samples of one or more subjects wherein a therapeutic treatment outcome of adverse side effects is demonstrated; (e) concentrating one or more microbial nucleic acid composition of said one or more subjects' one or more biological sample; (f) analyzing said one or more microbial nucleic acid compositions of said one or more subjects; (g) analyzing said one or more non-microbial nucleic acid compositions of said one or more subjects; (h) receiving one or more non-genomic data of said one or more subjects; (i) training an artificial intelligence wherein a training set comprises one or more microbial nucleic acid compositions, one or more non-microbial nucleic acid compositions, one or more non-genomic data and the therapeutic treatment outcome of one or more subjects; (j) generating a treatment outcome predictive model outcome with said trained artificial intelligence. In some embodiments, the method for a therapeutic treatment study data analysis system further comprises predicting the treatment outcome of an unknown subject with the trained artificial intelligence. In some embodiments, the one or more subjects are non-human mammal. In some embodiments, the one or more biological samples is a liquid biopsy. In some embodiments, the one or more biological samples is whole blood plasma, serum, saliva, sputum, urine, cerebral spinal fluid, sweat, tears, exhaled breath condensate, stool or any combination thereof. In some embodiments, the non-microbial nucleic acid composition comprises one or more single nucleotide polymorphisms (SNPs), insertions and/or deletions (INDELS), genomic amplifications and rearrangements. In some embodiments, the non-microbial nucleic acid compositions comprise cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA and/or methylation patterns of circulating tumor cell derived RNA or any combination thereof. In some embodiments, the non-genomic data comprises said one or more subjects gender, age, weight, body mass index, dietary factors, cardiovascular function, gastrointestinal function, immunological function, liver function, renal function, albumin concentration, alcohol intake, tobacco or marijuana use, pregnancy, lactation, exercise, comorbidities, occupational exposures, psychological status, and other medications or any combination thereof. In some embodiments, the therapeutic treatment is to treat cancer. In some embodiments, the microbial nucleic acid compositions comprise cell-free (cf) nucleic acids. In some embodiments, the microbial nucleic acid composition comprises microbial cell-free microbial DNA (cfDNA), cell-free microbial RNA (cfRNA) or any combination thereof. In some embodiments, the one or more microbial nucleic acid compositions originate from viral, bacterial, archaeal, fungal origin or any combination thereof. In some embodiments, the treatment outcome predictive model of the treatment outcome of the unknown subject is used to triage therapeutic clinical trial subjects into responder, non-responder, non-adverse, and adverse groups prior to therapeutic intervention. In some embodiments, the treatment outcome predictive model is used retrospectively. In some embodiments, the treatment outcome predictive model is utilized to longitudinally model the course of one or more cancers' response to therapy. In some embodiments, the treatment outcome predictive model is used to analyze the therapeutic outcome of subjects receiving treatment for acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, or uveal melanoma or any combination thereof. In some embodiments, the artificial intelligence is a machine learning model. In some embodiments, the artificial intelligence is a regularized machine learning model. In some embodiments, the artificial intelligence is a combination of one or more machine learning models. In some embodiments, the treatment outcome predictive model identifies and removes one or more microbial nucleic acids classified as noise while selectively retaining other one or more microbial features termed signal.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1C show an example predictive model training scheme and use of the trained model to identify signatures indicative of different possible response profiles. FIG. 1A illustrates an exemplary training structure of a predictive model with retrospective subjects' data and the use of such a trained model to predict therapeutic response or safety profile of subjects prior to administering a therapeutic agent. FIG. 1B. illustrates the use of a trained model of FIG. 1A to predict one or more subjects' response profiles based on the one or more subjects' one or more microbial and/or non-microbial nucleic acid compositions. FIG. 1C shows the use of the predictive model, generated in FIG. 1A, to identify signatures indicative of different possible response profiles based on the subjects' one or more microbial nucleic compositions, known therapeutic response or safety profiles, optionally their clinical metadata or any combination thereof, as described in some embodiments herein.

FIG. 2 illustrates a prospective prediction scheme for an ensemble of trained models, as described in some embodiments herein.

FIGS. 3A-3C show experimental data for filtering and generating predictive models with cell-free RNA sequencing data of subjects with ovarian cancer, as described in some embodiments herein.

FIG. 4 shows a computer system suitable for training and implementing the predictive model, described in some embodiments herein.

### DETAILED DESCRIPTION

Therapeutic outcomes may vary between individuals based on their respective genomic make-up. Particularly, mutations within a subject's genome altering enzyme metabolic activity to properly metabolize a given therapeutic could lead to severe toxicity, severe side effects or a potentially beneficial metabolic kinetic profile. In some aspects, a subject's genome encoding for such necessary enzymes may be influenced by external factors including but not limited to dietary factors, cardiovascular function, gastrointestinal function, immunologic function, liver function, renal function, albumin concentration, stress, fever, starvation, alcohol intake, tobacco or marijuana use (e.g., orally available and/or smoked), age, sex, pregnancy, lactation, exercise, sunlight exposure, presence or lack thereof disease, presence or lack thereof infection, occupational exposures, psychologic status, consumption of pharmaceutical and/or nutraceutical compounds, circadian and seasonal variations or any combination thereof. However, a subject's microbiome has not been explored as one such influential factor or as another possible direct influencer of pharmaceutical metabolism. Provided herein are methods and systems that may be configured to improve therapeutic outcomes by detecting circulating microbial and non-microbial nucleic acid compositions and correlating said microbial and non-microbial composition of a first set of one or more subjects undergoing therapeutic treatment with said one or more subjects' at least one intervention outcome to predict therapeutic efficacy and guide therapeutic administration of a second set of one or more subjects by their corresponding one or more microbial and non-microbial nucleic acid compositions.

The systems and methods disclosed herein may comprise a study data analysis system wherein the study data analysis system and methods may provide prospective or retrospective prediction of a subject's therapeutic response to a given compound in view of said subject's one or more microbial nucleic and non-microbial acid compositions. In some embodiments, the method may comprise determining a correlation between one or more subjects' one or more microbial nucleic acid compositions, one or more non-microbial nucleic acid compositions, non-genomic metadata, and a corresponding therapeutic outcome. In some embodiments, the one or more subjects may be non-human mammal. In some embodiments, the one or more subjects may be human. In some embodiments, the one or more microbial and/or non-microbial nucleic acid compositions may comprise one or more single nucleotide polymorphisms (SNPs), insertions and/or deletions (INDELS). In some embodiments, the one or more non-microbial nucleic acid compositions may comprise cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, or any combination thereof. In some embodiments, the treatment may comprise cancer treatments. In some embodiments, the cancer may comprise acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma or any combination thereof.

The methods and systems described herein, through the combined used of microbial and non-microbial nucleic acid compositions of a subject's liquid biopsy, provide better than expected predictive accuracy of therapeutic outcome of a given therapeutic treatment for a given subject. One of ordinary skill in the art, based on the developed scientific understanding of pharmacogenomics would routinely apply and seek to uncover associations between the human genome and variations thereof to measurable changes in pharmacokinetics of therapeutic compounds. The extent that microbial genomics has been found to influence pharmacogenomics, e.g., the diminished effect of chemotherapeutic compound gemcitabine by local tumor microbiota, is beyond the scope of routine scientific iterative experimentation. Such scientific realizations are not well understood and could not be realized with mere iterations upon the established scientific principles lending to the novelty and non-obviousness characterization of the methods and systems described herein.

### Isolation of Microbial and Non-Microbial Nucleic Acid Compositions

In some embodiments, the methods disclosed herein may comprise a method of isolating, concentrating, purifying and/or analyzing one or more liquid biopsies of one or more subjects. In some cases, the one or more liquid biopsies of one or more subjects may comprise one or more cell-free microbial and/or non-microbial nucleic acid compositions. In some cases, the one or more liquid biopsies may comprise human and/or non-human mammalian liquid biopsies. In some embodiments, the methods and systems described herein may process one or more human and/or non-human mammalian liquid biopsy samples e.g., to purify the liquid biopsy isolating the one or more cell-free microbial and/or non-microbial nucleic acid compositions from the remainder of the liquid biopsy. In some embodiments, the human and/or non-human mammalian liquid biopsy samples may comprise whole blood, plasma, serum, saliva, sputum, urine, cerebral spinal fluid, sweat, tears, exhaled breath condensate, stool or any combination thereof. In some embodiments, the liquid biopsy may require further processing to purify the liquid biopsy to isolate all one or more microbial or non-microbial nucleic acid material. In some embodiments, the one or more non-microbial nucleic acid compositions may comprise cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, or any combination thereof. In some embodiments, the one or more microbial nucleic acid compositions may originate from non-mammalian domains of life such as viral, bacterial, archaeal, fungal, or any combination thereof domains of life.

### Microbial Nucleic Acid Sequencing and Therapeutic Predictive Model Generation

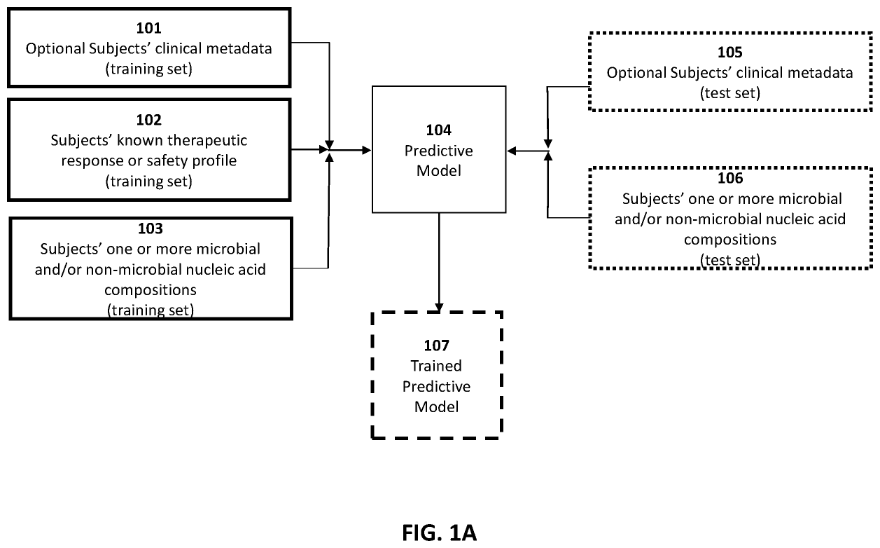

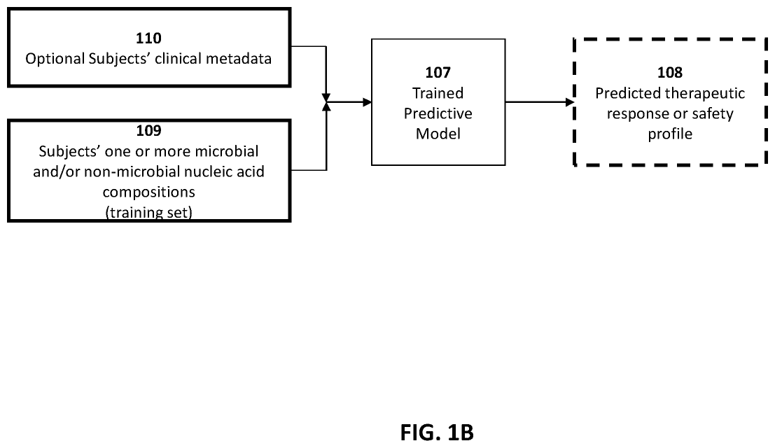

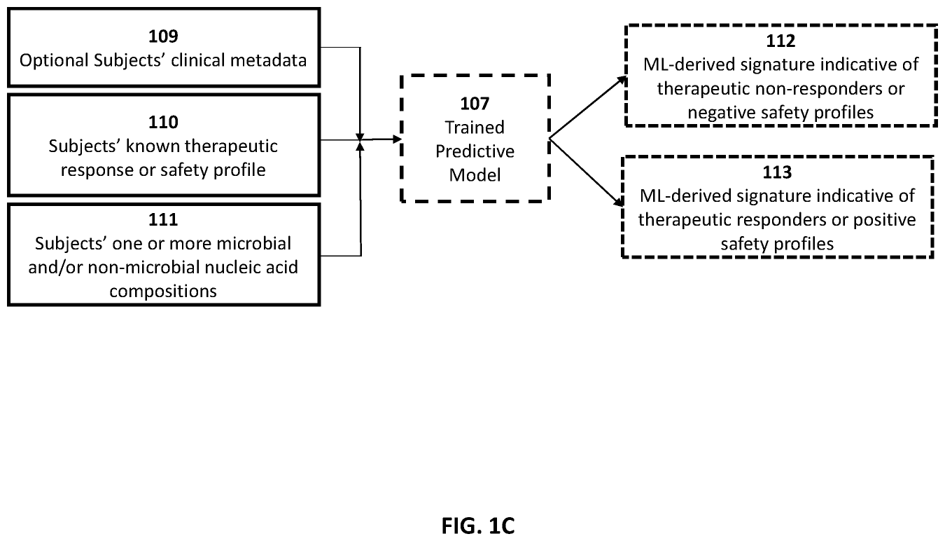

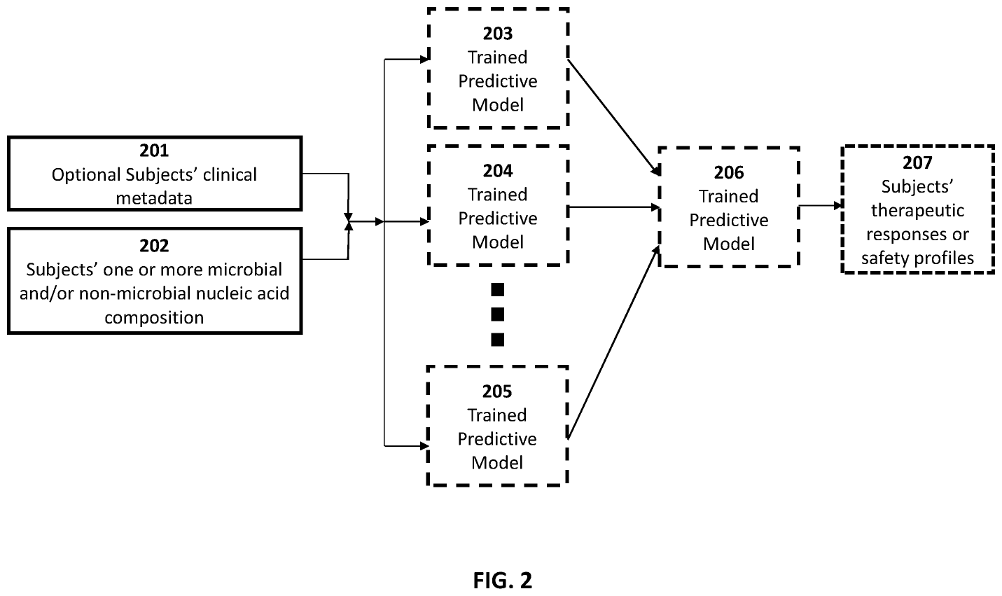

Upon purification of the one or more microbial and/or non-microbial nucleic acid compositions of the one or more liquid biopsies, described elsewhere herein, the systems and methods of the invention disclosed herein, in some embodiments, may comprise methods of processing and analyzing the one or more microbial and/or non-microbial nucleic acid compositions. In some cases, the method may comprise the steps of (a) sequencing the one or more microbial and/or non-microbial nucleic acid compositions thereby generating one or more microbial and/or non-microbial nucleic acid composition sequences; and (b) generating a predictive model with the one or more microbial and/or non-microbial nucleic acid composition sequences. In some embodiments, the sequencing method may comprise next-generation sequencing or long-read sequencing (i.e., third generation sequencing) or any combination thereof. In some embodiments, the predictive model may comprise a trained predictive model 107 as seen in FIGS. 1A-B. In some embodiments, the trained predictive model may comprise a trained machine learning model. In some embodiments, the predictive model may be a regularized machine learning model. In some embodiments, the trained machine learning model may comprise a linear regression, logistic regression, decision tree, support vector machine (SVM), naïve bayes, k-nearest neighbors (kNN), k-Means, random forest, or any combination thereof models. In some embodiments, the predictive model may be a combination of one or more trained predictive models 203, 204 and 205, as seen in FIG. 2. In some embodiments, the one or more predictive models' output may be further analyzed by another trained predictive model 206 to predict subjects' therapeutic responses or safety profiles 207. In some embodiments, the trained machine learning model may provide a retrospective analysis of subjects' one or more microbial and/or non-microbial nucleic acid sequences.

In some embodiments, the machine learning model may be trained with a retrospective training and validation data set of a first set of subjects' one or more cell free microbial and/or non-microbial nucleic acid sequences 103, subjects' known therapeutic response or safety profile to a given one or more therapeutic treatments 102 and/or optionally a subjects' clinical non-genomic metadata 101, as seen in FIG. 1A. In some embodiments, the therapeutic response, safety profile, or outcome for the therapeutic treatment may classify subjects as responders or non-responders. In some embodiments, the therapeutic response, safety profile, or outcome, may further classify subjects as non-adverse responders or adverse responders to a given therapeutic treatment. In some embodiments, the subjects' optional clinical non-genomic metadata may comprise subjects' gender, age, weight, body mass index, dietary factors, cardiovascular function, gastrointestinal function, immunological function, liver function, renal function, albumin concentration, alcohol intake, tobacco or marijuana use, pregnancy, lactation, exercise, comorbidities, occupational exposures, psychological status, other medications or any combination thereof. In some instances, the training set of subjects (101, 102, 103) and the test set subjects (105, 106) may be a same or different set of subjects. In some cases, the test-validation data ratio may comprise at least a 10:90, 20:80, 40:60, 50:50, 60:40, 70:30, 80:20, or 90:10 test:validation data set. In some instances, the test:validation data set ratio may comprise at most a 10:90, 20:80, 30:70, 40:60, 50:50, 70:30, 80:20, or 90:10 test:validation data set.

Aspects disclosed herein provide a method of training a predictive model (FIG. 1A) comprising: (a) providing as a training and validation data set comprising (i) one or more subjects' one or more sequenced microbial and/or non-microbial nucleic acid compositions 103; (ii) optionally one or more subjects' clinical metadata 101; and (iii) one or more subjects' therapeutic response to a given therapeutic treatment 102; (b) training the predictive model on a test-validation data set; and (c) evaluating the predictive accuracy of the predictive model.

In some embodiments, once the predictive model is trained, subjects' one or more microbial and/or non-microbial nucleic acid compositions 109 and optional clinic metadata 110 may be used as inputs into the trained predictive model 107 to predict a therapeutic response or safety profile 108, as seen in FIG. 1B. In some embodiments, the trained predictive model may provide a prospective and/or retrospective therapeutic response or safety profile prediction 108 to the input of subjects' one or more microbial and/or non-microbial nucleic acid compositions 109 and optionally subjects' clinical metadata 110.

In some embodiments, the prospective prediction 108 made by the trained predictive model 107 may comprise a machine learning signature indicative of therapeutic non-responders or negative safety profile 112, machine learning derived signature indicative of therapeutic responders or positive safety profiles 113 or any combination thereof as seen in FIG. 1C. In some embodiments, the trained predictive model may provide the ML-derived signatures 112, 113 or any combination thereof based on subjects' retrospective data including: (i) subjects' one or more microbial and/or non-microbial nucleic acid compositions 111, (ii) subjects' known therapeutic responses or safety profiles 110, (iii) subjects' optional clinical metadata 109 or any combination thereof. In some embodiments, the prospective prediction may comprise a response to at least the one therapeutic treatment. In some embodiments, the trained predictive model may identify and remove the one or more microbial and/or non-microbial nucleic acids classified as noise while selectively retaining other one or more microbial and/or non-microbial sequences termed signal. In some embodiments, the prospective prediction response may comprise subjects classified as responders or non-responders for one or more subject participants in a clinical trial. In some embodiments, the prospective prediction response may comprise subjects further classified as adverse or non-adverse responders for one or more subject participants in a clinical trial. In some embodiments, the therapeutic response or safety profile parameter of the predictive model may be utilized to provide longitudinal modeling of the course of one or more cancers' response to the treatment.

### Computer Systems

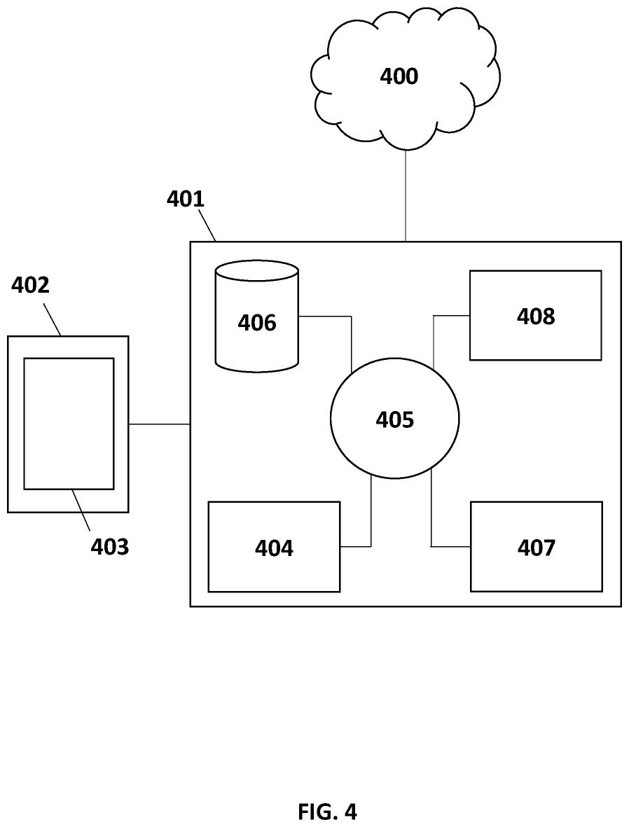

FIG. 4 shows a computer system 401 suitable for implementing and/or training the predictive models described herein. The computer system 401 may process various aspects of information of the present disclosure, such as, for example, subjects' one or more cell free microbial and/or non-microbial nucleic acid sequences, subjects' known therapeutic response or safety profile to a given one or more therapeutic treatments and/or subjects' clinical non-genomic metadata. The computer system 401 may be an electronic device. The electronic device may be a mobile electronic device.

The computer system 401 may comprise a central processing unit (CPU, also “processor” and “computer processor” herein) 405, which may be a single core or multi core processor, or a plurality of processors for parallel processing. The computer system 401 may further comprise memory or memory locations 404 (e.g., random-access memory, read-only memory, flash memory), electronic storage unit 406 (e.g., hard disk), communications interface 408 (e.g., network adapter) for communicating with one or more other devices, and peripheral devices 407, such as cache, other memory, data storage and/or electronic display adapters. The memory 404, storage unit 406, interface 408, and peripheral devices 407 are in communication with the CPU 405 through a communication bus (solid lines), such as a motherboard. The storage unit 406 may be a data storage unit (or a data repository) for storing data. The computer system 401 may be operatively coupled to a computer network (“network”) 400 with the aid of the communication interface 408. The network 400 may be the Internet and/or extranet, or an intranet and/or extranet that is in communication with the Internet. The network 400 may, in some cases, be a telecommunication and/or data network. The network 400 may include one or more computer servers, which may enable distributed computing, such as cloud computing. The network 400, in some cases with the aid of the computer system 401, may implement a peer-to-peer network, which may enable devices coupled to the computer system 401 to behave as a client or a server.

The CPU 405 may execute a sequence of machine-readable instructions, which may be embodied in a program or software. The instructions may be directed to the CPU 405, which may subsequently program or otherwise configured the CPU 405 to implement methods of the present disclosure. Examples of operations performed by the CPU 405 may include fetch, decode, execute, and writeback.

The CPU 405 may be part of a circuit, such as an integrated circuit. One or more other components of the system 401 may be included in the circuit. In some cases, the circuit is an application specific integrated circuit (ASIC).

The storage unit 406 may store files, such as drivers, libraries and saved programs. The storage unit 406 may store subjects' one or more cell free microbial and/or non-microbial nucleic acid sequences, subjects' known therapeutic response or safety profile to a given one or more therapeutic treatments and/or subjects' clinical non-genomic metadata. The computer system 401, in some cases may include one or more additional data storage units that are external to the computer system 401, such as located on a remote server that is in communication with the computer system 401 through an intranet or the internet.

Methods as described herein may be implemented by way of machine (e.g., computer processor) executable code stored on an electronic storage location of the computer device 401, such as, for example, on the memory 404 or electronic storage unit 406. The machine executable or machine-readable code may be provided in the form of software. During use, the code may be executed by the processor 405. In some instances, the code may be retrieved from the storage unit 406 and stored on the memory 404 for ready access by the processor 405. In some instances, the electronic storage unit 406 may be precluded, and machine-executable instructions are stored on memory 404.

The code may be pre-compiled and configured for use with a machine having a processor adapted to execute the code or may be compiled during runtime. The code may be supplied in a programming language that may be selected to enable the code to be executed in a pre-complied or as-compiled fashion.

Aspects of the systems and methods provided herein, such as the computer system 401, may be embodied in programming. Various aspects of the technology may be thought of a “product” or “articles of manufacture” typically in the form of a machine (or processor) executable code and/or associated data that is carried on or embodied in a type of machine readable medium. Machine-executable code may be stored on an electronic storage unit, such memory (e.g., read-only memory, random-access memory, flash memory) or a hard disk. “Storage” type media may include any or all of the tangible memory of a computer, processor the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, term such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media may include, for example, optical or magnetic disks, such as any of the storage devices in any computer(s) or the like, such as may be used to implement the databases, etc. Volatile storage media include dynamic memory, such as main memory of such a computer platform. Tangible transmission media includes coaxial cables; copper wire and fiber optics, including the wires that comprise a bus within a computer device. Carrier-wave transmission media may take the form of electric or electromagnetic signals, or acoustic or light waves such as those generated during radio frequency (RF) and infrared (IR) data communications. Common forms of computer-readable media therefor include for example: a floppy disk, a flexible disk, hard disk, magnetic tape, any other magnetic medium, a CD-ROM, DVD or DVD-ROM, any other optical medium, punch cards paper tape, any other physical storage medium with pattern of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one more instruction to a processor for execution.

The computer system may include or be in communication with an electronic display 402 that comprises a user interface (UI) 403 for inputting donor parameters and viewing the association of a donor's parameters with a generated model. Examples of UI's include, without limitation, a graphical user interface (GUI) and web-based user interface.

Methods and systems of the present disclosure can be implemented by way of one or more algorithms and with instructions provided with one or more processors as disclosed herein. An algorithm can be implemented by way of software upon execution by the central processing unit 405. The algorithm can be, for example, random forest, graphical models, support vector machine or other.

Although the above steps show a method of a system in accordance with an example, a person of ordinary skill in the art will recognize many variations based on the teaching described herein. The steps may be completed in a different order. Steps may be added or deleted. Some of the steps may comprise sub-steps. Many of the steps may be repeated as often as if beneficial to the platform.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

Any systems, methods, software, compositions, and platforms described herein are modular and not limited to sequential steps. Accordingly, terms such as “first” and “second” do not necessarily imply priority, order of importance, or order of acts.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### EMBODIMENTS

1. A method for generating a predictive model, comprising:
   (a) receiving:
      i. one or more liquid biopsies of a first set of one or more subjects, wherein said one or more liquid biopsies comprise one or more microbial and non-microbial nucleic acid compositions; and
      ii. at least one therapeutic outcome of said first set of one or more subjects administered a therapeutic treatment;
   (b) determining one or more sequences of said one or more microbial and non-microbial nucleic acid compositions of said first set of one or more subjects;
   (c) determining a correlation between said first set of one or more subjects' said at least one therapeutic outcome and said one or more sequences of said one or more microbial and non-microbial nucleic acid compositions; and
   (d) generating a predictive model with said correlation, wherein said predictive model is configured to provide a prediction of at least one therapeutic outcome of a second set of one or more subjects when administered said therapeutic treatment based on an input to said predictive model of said second set of one or more subjects' one or more microbial and non-microbial nucleic acid composition one or more sequences, wherein said first set of one or more subjects is different than said second set of one or more subjects.
2. The method of embodiment 1, wherein said first or second set of one or more subjects' said at least one therapeutic outcome comprises therapeutic efficacy, therapeutic failure, therapeutic safety, therapeutic adverse side effect or any combination thereof
3. The method of embodiment 1, wherein said first or second set of one or more subjects' one or more microbial nucleic acid compositions comprise cell-free (cf) nucleic acids.
4. The method of embodiment 1, wherein said first or second set of one or more subjects' one or more microbial nucleic acid compositions comprise microbial cell-free microbial DNA (cf-mbDNA), cell-free microbial RNA (cf-mbRNA) or any combination thereof
5. The method of embodiment 1, wherein said first or second set of one or more subjects' one or more microbial nucleic acid compositions comprise an origin of viral, bacterial, archaeal, fungal sources or any combination thereof
6. The method of embodiment 1, wherein said therapeutic treatment treats cancer.
7. The method of embodiment 1, wherein said predictive model is used to analyze a therapeutic outcome of subjects treated for acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, or uveal melanoma or any combination thereof
8. The method of embodiment 1, wherein said predictive model comprises an artificial intelligence machine learning model, wherein said artificial intelligence machine learning model is trained with said one or more microbial and non-microbial nucleic acid composition said one or more sequences of said first set of one or more subjects and said correlation between said first set of one or more subjects' said one or more microbial and non-microbial nucleic acid composition said one or more sequences and said at least one therapeutic outcome.
9. The method of embodiment 1, wherein said therapeutic outcome of said second set of one or more subjects is used to triage therapeutic clinical trial subjects into responder, non-responder, non-adverse, and adverse groups prior to therapeutic intervention.
10. The method of embodiment 1, wherein said predictive model is used retrospectively.
11. The method of embodiment 1, wherein said second set of one or more subjects' said at least one therapeutic outcome of said predictive model is utilized to longitudinally model the course of one or more cancers' response to therapy.
12. The method of embodiment 1, wherein said first or second set of one or more subjects' one or more microbial or non-microbial nucleic acid compositions comprise one or more single nucleotide polymorphisms (SNPs), insertions and/or deletions (INDELS), genomic amplifications and rearrangements or any combination thereof.
13. The method of embodiment 1, wherein said first or second set of one or more subjects' one or more non-microbial nucleic acid compositions comprise cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA and/or methylation patterns of circulating tumor cell derived RNA, or any combination thereof.
14. The method of embodiment 1, wherein receiving further comprises said first set of index, dietary factors, cardiovascular function, gastrointestinal function, immunological function, liver function, renal function, albumin concentration, alcohol intake, tobacco or marijuana use, pregnancy, lactation, exercise, comorbidities, occupational exposures, psychological status, other medications, or any combination thereof.
15. The method of embodiment 1, wherein said predictive model is a machine learning model.
16. The method of embodiment 1, wherein said predictive model is a regularized machine learning model.
17. The method of embodiment 1, wherein said predictive model is a combination of one or more machine learning models.
18. The method of embodiment 1, wherein said predictive model identifies and removes said first or second one or more subjects' one or more microbial or non-microbial nucleic acid compositions classified as noise while selectively retaining other said one or more microbial or non-microbial features termed signal.
19. The method of embodiment 1, wherein said first or second set of one or more subjects are non-human mammal.
20. The method of embodiment 1, wherein said first or second set of one or more subjects are human.
21. The method of embodiment 1, wherein said liquid biopsy comprises whole blood, plasma, serum, saliva, sputum, urine, cerebral spinal fluid, sweat, tears, exhaled breath condensate, stool, or any combination thereof.
22. A method for generating a therapeutic treatment prediction of one or more subjects, comprising:
   (a) providing one or more biological samples of a first set of one or more subjects, wherein said first set of one or more subjects comprise a therapeutic treatment outcome when administered a therapeutic treatment;
   (b) sequencing said one or more microbial and non-microbial nucleic acid compositions of said first set of one or more subjects thereby generating one or more sequences;
   (c) training a predictive model with said first set of one or more subjects' microbial and non-microbial nucleic acid compositions said one or more sequences and said therapeutic treatment outcome, thereby producing a trained predictive model; and
   (d) generating a therapeutic treatment prediction for a second set of one or more subjects by inputting said second set of one or more subjects' one or more microbial and non-microbial nucleic compositions one or more sequences, clinical meta data, and said therapeutic treatment to be administered into said predictive model.
23. The method of embodiment 22, wherein said therapeutic treatment treats cancer.
24. The method of embodiment 22, wherein said first or second set of one or more subjects' one or more microbial nucleic acid compositions comprise cell-free (cf) nucleic acids.
25. The method of embodiment 22, wherein said first or second set of one or more subjects' one or more microbial nucleic acid compositions comprise microbial cell-free microbial DNA (cf-mbDNA), cell-free microbial RNA (cf-mbRNA), or any combination thereof
26. The method of embodiment 22, wherein said first or second set of one or more subjects' one or more microbial nucleic acid compositions originate from viral, bacterial, archaeal, fungal sources or any combination thereof.
27. The method of embodiment 22, wherein said therapeutic treatment prediction is used to triage therapeutic clinical trial subjects into responder, non-responder, non-adverse, and adverse groups prior to therapeutic intervention.
28. The method of embodiment 22, wherein said trained predictive model is used retrospectively.
29. The method of embodiment 22, wherein said trained predictive model is utilized to longitudinally model the course of one or more cancers' response to therapy.
30. The method of embodiment 22, wherein said predictive model is used to analyze said therapeutic treatment prediction of one or more subjects receiving treatment for acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, or uveal melanoma or any combination thereof.
31. The method of embodiment 22, wherein said predictive model comprises a machine learning model.
32. The method of embodiment 31, wherein said machine learning model is a regularized machine learning model.
33. The method of embodiment 31, wherein said machine learning model is a combination of one or more machine learning models.
34. The method of embodiment 22, wherein said predictive model identifies and removes said first or second one or more subjects' one or more microbial or non-microbial nucleic acids classified as noise while selectively retaining other said one or more microbial or non-microbial features termed signal.
35. The method of embodiment 22, wherein said first or second set of one or more subjects are non-human mammal.
36. The method of embodiment 22, wherein said first or second set of one or more subjects are human.
37. The method of embodiment 22, wherein said one or more biological samples are liquid biopsies.
38. The method of embodiment 22, wherein said one or more biological samples is whole blood, plasma, serum, saliva, sputum, urine, cerebral spinal fluid, sweat, tears, exhaled breath condensate, stool or any combination thereof
39. The method of embodiment 22, wherein said first or second set of one or more subjects' treatment outcome comprise efficacy, failure, safety, adverse side effects, or any combination thereof.
40. The method of embodiment 22, further comprising concentrating said first or second set of one or more subjects' one or more microbial and non-microbial nucleic acid compositions of said first or second set of subjects' one or more biological samples.
41. A method of generating a predictive model, comprising:
   (a) receiving:
      i. one or more liquid biopsies of one or more subjects comprising one or more microbial nucleic acid compositions; and
      ii. at least one therapeutic outcome of said one or more subjects undergoing treatment in a clinical trial;
   (b) analyzing one or more sequences of said one or more microbial nucleic acid compositions of said one or more subjects; and
   (c) generating a predictive model with said one or more sequences of said one or more microbial nucleic acid compositions and said at least one therapeutic outcome of said one or more subjects.
42. The method of embodiment 41, wherein said at least one therapeutic outcome comprises therapeutic efficacy, therapeutic failure, therapeutic safety, therapeutic adverse side effect, or any combination thereof.
43. The method of embodiment 41, wherein said one or more microbial nucleic acid compositions comprise cell-free (cf) nucleic acids.
44. The method of embodiment 41, wherein said one or more microbial nucleic acid compositions comprise microbial cell-free microbial DNA (cf-mbDNA), cell-free microbial RNA (cf-mbRNA), or any combination thereof
45. The method of embodiment 41, wherein said one or more microbial nucleic acid compositions comprise an origin of viral, bacterial, archaeal, fungal sources, or any combination thereof
46. The method of embodiment 41, wherein said treatment treats cancer.
47. The method of embodiment 41, wherein said predictive model is configured to analyze said therapeutic outcome of subjects treated for acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, or uveal melanoma, or any combination thereof
48. The method of embodiment 41, wherein said predictive model is configured to triage therapeutic clinical trial subjects into responder, non-responder, non-adverse, and adverse groups prior to therapeutic intervention.
49. The method of embodiment 41, wherein said predictive model is used retrospectively.
50. The method of embodiment 41, wherein said predictive model is configured to provide at least one therapeutic outcome prediction in response to a second set of subjects' one or more microbial nucleic acid compositions' one or more sequences to longitudinally model the course of one or more cancers' response to said treatment.
51. The method of embodiment 41, wherein said one or more liquid biopsies further comprise non-microbial nucleic acid compositions, wherein said one or more microbial or non-microbial nucleic acid compositions comprise one or more single nucleotide polymorphisms (SNPs), insertions and/or deletions (INDELS), genomic amplifications and rearrangements or any combination thereof.
52. The method of embodiment 51, wherein said one or more non-microbial nucleic acid compositions comprise cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA and/or methylation patterns of circulating tumor cell derived RNA or any combination thereof
53. The method of embodiment 41, wherein receiving further comprises said one or more subjects' non-genomic data comprising gender, age, weight, body mass index, dietary factors, cardiovascular function, gastrointestinal function, immunological function, liver function, renal function, albumin concentration, alcohol intake, tobacco or marijuana use, pregnancy, lactation, exercise, comorbidities, occupational exposures, psychological status, other medications, or any combination thereof.
54. The method of embodiment 41, wherein said predictive model is a machine learning model.
55. The method of embodiment 41, wherein said predictive model is a regularized machine learning model.
56. The method of embodiment 41, wherein said predictive model is a combination of one or more machine learning models.
57. The method of embodiment 41, wherein said predictive model identifies and removes said one or more subjects' one or more microbial nucleic acid compositions classified as noise while selectively retaining other said one or more microbial features termed signal.
58. The method of embodiment 41, wherein said one or more subjects are non-human mammal.
59. The method of embodiment 41, wherein said one or more subjects are human.
60. The method of embodiment 41, wherein said one or more liquid biopsies comprise whole blood, plasma, serum, saliva, sputum, urine, cerebral spinal fluid, sweat, tears, exhaled breath condensate, stool, or any combination thereof.
61. The method of embodiment 51, further comprising determining a correlation between said one or more subjects' said at least one therapeutic outcome and said one or more sequences of said one or more microbial or non-microbial nucleic acid compositions.
62. A computer-implemented method for utilizing a predictive model to provide a therapeutic treatment prediction for one or more subjects, the method comprising:
   (a) receiving a first set of one or more subjects' one or more liquid biopsies comprising one or more non-microbial and microbial nucleic acid compositions genetic sequences and corresponding at least one therapeutic outcome of said first set of one or more subjects' when exposed to a treatment;
   (b) training a predictive model with said genetic sequences and said corresponding therapeutic responses of said first set of one or more subjects, thereby generating a trained predictive model; and
   (c) outputting a therapeutic treatment prediction using said trained predictive model when inputted with a second set of one or more subjects' microbial and non-microbial nucleic acid sequences and corresponding treatment to be administered.
63. The method of embodiment 62, wherein said first or second set of one or more subjects' at least one therapeutic outcome comprises therapeutic efficacy, therapeutic failure, therapeutic safety, therapeutic adverse side effect or any combination thereof
64. The method of embodiment 62, wherein said first or second set of one or more subjects' one or more microbial nucleic acid compositions comprise cell-free (cf) nucleic acids.
65. The method of embodiment 62, wherein said first or second set of one or more subjects' one or more microbial nucleic acid compositions comprise microbial cell-free microbial DNA (cf-mbDNA), cell-free microbial RNA (cf-mbRNA) or any combination thereof
66. The method of embodiment 62, wherein said first or second set of one or more subjects' one or more microbial nucleic acid compositions comprise an origin of viral, bacterial, archaeal, fungal sources or any combination thereof
67. The method of embodiment 62, wherein said treatment treats cancer.
68. The method of embodiment 62, wherein said predictive model is configured to analyze a therapeutic outcome of subjects treated for acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, or uveal melanoma or any combination thereof.
69. The method of embodiment 62, wherein said predictive model is configured to triage therapeutic clinical trial subjects into responder, non-responder, non-adverse, and adverse groups prior to therapeutic intervention.
70. The method of embodiment 62, wherein said predictive model is used retrospectively.
71. The method of embodiment 62, wherein said second set of one or more subjects' at least one therapeutic outcome of said predictive model is utilized to longitudinally model the course of one or more cancers' response to therapy.
72. The method of embodiment 62, wherein said first or second set of one or more subjects' microbial or non-microbial nucleic acid compositions comprise one or more single nucleotide polymorphisms (SNPs), insertions and/or deletions (INDELS), genomic amplifications and rearrangements, or any combination thereof.
73. The method of embodiment 62, wherein said first or second set of one or more subjects' one or more non-microbial nucleic acid compositions comprise cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA and/or methylation patterns of circulating tumor cell derived RNA, or any combination thereof.
74. The method of embodiment 62, wherein receiving further comprises said first set of index, dietary factors, cardiovascular function, gastrointestinal function, immunological function, liver function, renal function, albumin concentration, alcohol intake, tobacco or marijuana use, pregnancy, lactation, exercise, comorbidities, occupational exposures, psychological status, other medications, or any combination thereof.
75. The method of embodiment 62, wherein said predictive model is a machine learning model.
76. The method of embodiment 62, wherein said predictive model is a regularized machine learning model.
77. The method of embodiment 62, wherein said predictive model is a combination of one or more machine learning models.
78. The method of embodiment 62, wherein said predictive model identifies and removes said first or second set of one or more subjects' one or more microbial nucleic acid compositions classified as noise while selectively retaining other said one or more microbial features termed signal.
79. The method of embodiment 62, wherein said first or second set of one or more subjects are non-human mammal.
80. The method of embodiment 62, wherein said first or second set of one or more subjects are human.
81. The method of embodiment 62, wherein said one or more liquid biopsies comprises whole blood, plasma, serum, saliva, sputum, urine, cerebral spinal fluid, sweat, tears, exhaled breath condensate, stool or any combination thereof
82. The method of embodiment 62, further comprising determining a correlation between said first set of one or more subjects' said at least one therapeutic outcome and said one or more sequences of said one or more microbial and non-microbial nucleic acid compositions.

### EXAMPLES

Each of the examples as described herein can be combined with one or more other examples. Further, one or more components of one or more examples can be combined with other examples.

### Example 1: Generating a Predictive Model to Determine Subjects with Favorable Therapy Response from their Cell Free Microbiota

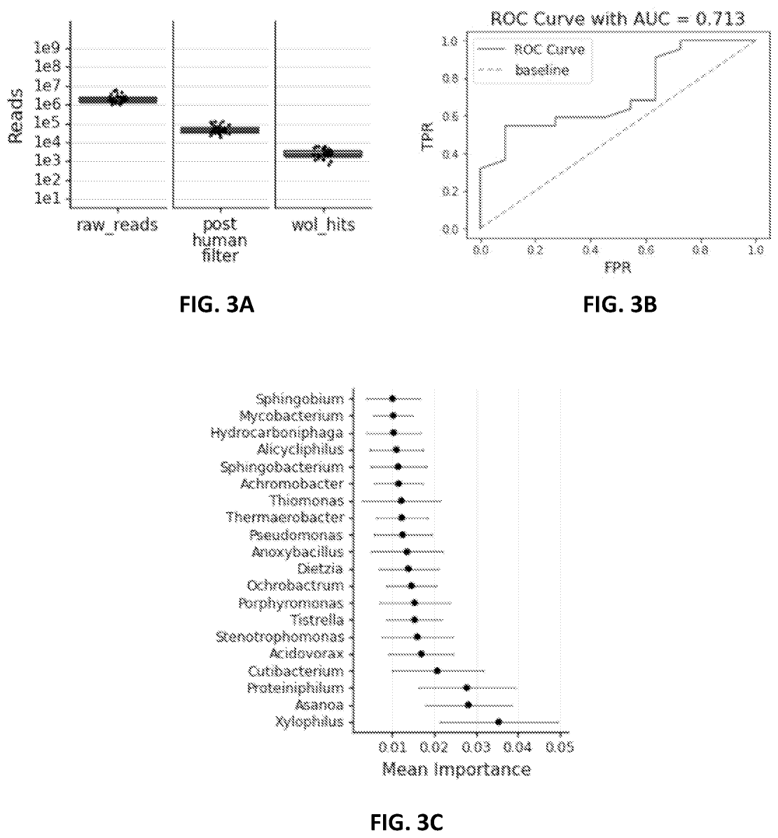

Serum cell free RNA (cfRNA) sequencing data from 33 subjects with ovarian cancer (https://www.ebi.ac.uk/ena/browser/view/PRJNA517159?show=xrefs) was utilized to generate a predictive model capable of predicting which subjects will respond favorably or have a poor outcome when provided a therapeutic intervention. Prior to model generation the 33 subjects' (22 favorable, 11 poor outcome to the intervention) cfRNA was sequenced (about 1 million paired reads/sample) and labeled with the intervention outcome for the given subject. Next, the sequenced reads of the 33 subjects were subjected to filtering and processing, the result thereof shown in FIG. 3A. The sequencing data of the 33 subjects' cfRNA was then aligned and filtered against a human genome database (e.g., Genome Reference Consortium Human GRCh38) to remove all human mammalian sequencing reads, yielding about 2700 hits per sample. The non-mammalian cfDNA sequencing reads were taxonomically assigned via query against a microbial reference genome database (e.g., Web of Life (WOL)) and, lastly, the taxonomically classified reads were filtered against a library of known contaminant non-mammalian microbes, virus, etc., to yield decontaminated sequencing reads and their associated abundances. The decontaminated sequencing reads were then used to train one or more predictive models with leave one out cross-validation. The trained predictive model provided a receiver operating characteristic area under the curve of 0.713 (FIG. 3B) in distinguishing favorable vs poor outcome subjects after receiving the therapeutic intervention. The top 20 features for the predictive model were determined to be Sphingobium, Mycobacterium, Hydrocarboniphaga, Alicycliphilus, Sphingobacterium, Achromobacter, Thiomonas, Thermaerobacter, Pseudomonas, Anoxybacillus, Dietzia, Ochrobacrum, Porphyromonas, Tistrella, Stenotrophomonas, Acidovorax, Cutibacterium, Proteiniphilum, Asanoa, and Xylophilus, as seen in FIG. 3C.

### Example 2: Generating a Predictive Model with Subjects' Microbial and Non-Microbial Nucleic Acid Compositions

A combined dataset of microbial and non-microbial nucleic acid compositions of one or more subjects' liquid biopsies are utilized to improve the predictive accuracy of the predictive models in determining subjects' response to a given one or more therapeutic compounds. Subjects', treated with one or more therapeutic compounds, one or more therapeutic microbial and non-microbial nucleic acid compositions are determined from one or more liquid biopsy samples using traditional in-vitro laboratory procedures to isolate and sequence the one or more microbial and non-microbial nucleic acid compositions from the remainder of the liquid biopsy constituents. Alternatively, or in addition to, subjects' microbial and non-microbial nucleic acid compositions one or more sequences, are accessed from a database to train one or more predictive models, described elsewhere herein. One or more predictive models are trained and tested with a first set of subjects' microbial and non-microbial one or more sequences, therapeutic response and/or safety profiles to one or more therapeutics, and clinical metadata. The one or more predictive models are trained with test:validation data set ratios of at least a 10:90, 20:80, 30:70, 40:60, 50:50, 60:40, 70:30, 80:20, or 90:10 test:validation data set. The one or more predictive models are then used to provide a prediction of a second set of one or more subjects' therapeutic response to one or more therapeutics and/or safety profiles by inputting the subjects' one or more microbial and non-microbial nucleic acid compositions and/or the subjects' meta-data into the trained predictive model.

1-82. (canceled)

83. A method for generating a therapeutic treatment prediction of one or more subjects, comprising:
(a) receiving:
i. one or more liquid biopsies of one or more subjects, wherein said one or more liquid biopsies comprise one or more microbial and non-microbial nucleic acid compositions;
ii. at least one therapeutic treatment of said one or more subjects; and
iii. clinical metadata from said one or more subjects;
(b) determining one or more sequences of said one or more microbial and non-microbial nucleic acid compositions of said one or more subjects; and
(c) processing said one or more microbial and non-microbial nucleic acid compositions and said clinical metadata with a trained predictive model thereby generating said therapeutic treatment prediction of said one or more subjects;
wherein said trained predictive model is trained with a plurality of microbial and non-microbial nucleic acid compositions derived from a liquid biopsy sample of subjects with a corresponding therapeutic outcome and clinical metadata from said subjects.

84. The method of claim 83; wherein said one or more subjects' said at least one therapeutic outcome comprises therapeutic efficacy; therapeutic failure; therapeutic safety; therapeutic adverse side effect; or any combination thereof.

85. The method of claim 83; wherein said one or more subjects' one or more microbial nucleic acid compositions comprise cell-free nucleic acids.

86. The method of claim 83, wherein said one or more subjects' one or more microbial nucleic acid compositions comprise microbial cell-free microbial DNA, cell-free microbial RNA, or any combination thereof.

87. The method of claim 83, wherein said one or more subjects' one or more microbial nucleic acid compositions comprise an origin of viral, bacterial, archaeal, fungal sources, or any combination thereof.

88. The method of claim 83, wherein said therapeutic treatment treats cancer.

89. The method of claim 83, wherein said predictive model is used to analyze a therapeutic outcome of subjects treated for acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof.

90. The method of claim 83, wherein said predictive model comprises an artificial intelligence machine learning model, wherein said artificial intelligence machine learning model is trained with said one or more microbial and non-microbial nucleic acid composition said one or more sequences of said one or more subjects and said correlation between said one or more subjects' said one or more microbial and non-microbial nucleic acid composition said one or more sequences and said at least one therapeutic outcome.

91. The method of claim 83, wherein said therapeutic outcome of said one or more subjects is used to triage therapeutic clinical trial subjects into responder, non-responder, non-adverse, and adverse groups prior to therapeutic intervention.

92. The method of claim 83, wherein said trained predictive model is used retrospectively.

93. The method of claim 83, wherein said one or more subjects' said at least one therapeutic outcome of said trained predictive model is utilized to longitudinally model the course of one or more cancers' response to therapy.

94. The method of claim 83, wherein said one or more subjects' one or more microbial or non-microbial nucleic acid compositions comprise one or more single nucleotide polymorphisms, insertions, deletions, genomic amplifications, rearrangements, or any combination thereof.

95. The method of claim 83, wherein said one or more subjects' one or more non-microbial nucleic acid compositions comprise cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA and/or methylation patterns of circulating tumor cell derived RNA, or any combination thereof.

96. The method of claim 83, further comprising receiving said one or more subjects' non-genomic data comprising gender, age, weight, body mass index, dietary factors, cardiovascular function, gastrointestinal function, immunological function, liver function, renal function, albumin concentration, alcohol intake, tobacco or marijuana use, pregnancy, lactation, exercise, comorbidities, occupational exposures, psychological status, other medications, or any combination thereof.

97. The method of claim 83, wherein said predictive model is a machine learning model, a regularized machine learning model, or a combination thereof.

98. The method of claim 83, wherein said predictive model is a combination of one or more machine learning models.

99. The method of claim 83, wherein said predictive model identifies and removes said one or more subjects' one or more microbial or non-microbial nucleic acid compositions classified as noise while selectively retaining other one or more microbial or non-microbial features termed signal.

100. The method of claim 83, further comprising determining taxonomic assignments from said one or more sequences of said one or more microbial nucleic acid compositions.

101. The method of claim 83, wherein said one or more subjects are human or non-human mammals.

102. The method of claim 83, wherein said liquid biopsy comprises whole blood, plasma, serum, saliva, sputum, urine, cerebral spinal fluid, sweat, tears, exhaled breath condensate, stool, or any combination thereof.
